(19) United States
(12) Patent Application Publication
(10) Pub. No.: US 2013/0296437 A1
(43) Pub. Date: Nov. 7, 2013

(54) Treatment of Depression

(71) Applicant: E-THERAPEUTICS PLC, Oxfordshire (GB)

(72) Inventors: Malcolm Philip Young, Hexham (GB); Philip McKeown, Gateshead (GB)

(73) Assignee: e-Therapeutics plc, Oxfordshire (GB)

(21) Appl. No.: 13/785,874

(22) Filed: Mar. 5, 2013

Related U.S. Application Data
(60) Provisional application No. 61/642,737, filed on May 4, 2012.

(51) Int. Cl. A61K 31/135

(52) U.S. Cl. CPC A61K 31/135
USPC 514/646

(57) ABSTRACT

There is described a method of treatment of a patient suffering from depression, said method comprising the administration of a sub-analgesic amount of tramadol, or a salt thereof.

### CROSS-REFERENCE TO RELATED APPLICATIONS

This application claims priority to and the benefit of U.S. provisional patent application 61/642,737, filed on May 4, 2012, the entire contents of which are hereby incorporated by reference herein.

### FIELD OF THE INVENTION

The present invention provides a novel composition for the treatment of depression and methods related thereto.

More particularly, compositions comprising tramadol as active ingredient for the treatment of depression, especially in the sub-set of patients which do not respond to conventional SSRI therapy (SSRI non-responders) and controlled release pharmaceutical compositions related thereto.

### BACKGROUND OF THE INVENTION

Statistics suggest that in the USA about 9 million people suffer from depression and about one in six people in the. UK will experience depression during their lifetime. Globally the figure is estimated at 340 million people. In both cases the figures are expected to increase, so that by 2020 depression is expected to be the second most disabling condition.

One of the major groups of medicines for the treatment of depression is Selective Serotonin Reuptake Inhibitors (SSRIs). SSRIs work by altering the amount of serotonin in the brain called

SSRIs are typically administered as antidepressants in the treatment of depression, anxiety disorders and some personality disorder. Well known SSRIs include citalopram, escitalopram, fluoxetine (Prozac®) paroxetine and sertraline. According to the MHRA, in England the number of SSRI prescriptions rose from 8.2 million in 1999 to over 19 million in 2003.

However, whilst the SSRIs have become the standard first-line treatment for all forms of clinical depression, at least 40%, varying from 40 to 60%, of patients fail to achieve a 50% reduction in depressive symptoms or have an adequate or satisfactory response to SSRI treatment. This group of patients are often referred to as “non-responders” In such cases a patient may be switched to an antidepressant of another class, although it is estimated that about 50% of patients who fail to respond to SSRI.

Given the large proportion of patients who do not respond adequately to SSRIs as first-line therapy, the medical practitioner is faced with the dilemma of determining the presence of inadequacy of the response and then selecting a new course of action.

The new course of action may vary and can include:
1) An optimization strategy (altering dose or duration of the SSRI).
2) Switching to another SSRI.
3) Switching to another class of antidepressants.
4) Combining an SSRI with another medication or non-pharmacological therapy.
5) Switching to a non-pharmacological intervention alone.
6) A combination of the above.

Switching to another SSRI will generally not prove to be successful. When a non-responding patient is switched to an alternative class of antidepressant, it will generally be selected from the group consisting of a tricyclic antidepressant (TCA), bupropion or a monoamine oxidase inhibitor (MAOI). However, all of these drugs are known to have significant undesirable adverse effects.

Therefore, there is a clear need for a therapeutically effect antidepressants with minimal adverse effects and especially one that is suited for the group of non-responders as hereinbefore described.

Tramadol is (1R,2R)-rel-2-[(dimethylamino)methyl]-1-(3-methoxyphenyl)cyclohexanol and is a centrally acting opioid analgesic that has been shown to be effective in a variety of acute and chronic pain states, including moderate and severe pain, either alone or in conjunction with non-steroidal anti-inflammatory drugs (NSAIDs) for the reduction of pain attributed to osteoarthritis.

In the treatment or management of moderate to moderately severe chronic pain in adults tramadol, usually as tramadol hydrochloride, will generally be administered as an analgesic at a dose range of from 100 mg to 400 mg per day, usually a dose of 50 mg to 100 mg four times a day. Thus, the analgesic therapeutic daily dose of tramadol is considered to be 100 mg for an adult.

International Patent application No. WO 2009/001040 describes certain compounds which are useful for the treatment or alleviation of depression contributed to or caused by pain. More particularly, the aforementioned application describes a compound selected from the group comprising tramadol, resveratrol, acetaminophen, xorphanol, cinfenoac, furcloprofen, bismuth subsalicylate, enofelast, triflusal, ketorfanol, indriline, furofenac, cizolirtine, dacemazine, demelverine, and fenethazine, and derivatives and/or combinations thereof, as being useful for the treatment or alleviation of depression; and especially depression contributed to or caused by pain.

We have now surprisingly found that low doses, i.e. doses previously thought to be sub-analgesic with little or no therapeutic benefit in the treatment of pain, are suitable for the treatment of depression and especially, but not limited to, the treatment of depression in the patient group of non-responders.

### SUMMARY OF THE INVENTION

The use of a sub-analgesic dose of tramadol, or a salt thereof in the treatment, alleviation or prevention of depression in a patient is novel.

Therefore, according to a first aspect of the invention there is provided a sub-analgesic amount of tramadol, or a salt thereof, in the treatment, alleviation or prevention of depression in a patient.

The term “sub-analgesic” amount should be understood to mean a dose that would conventionally be below the conventionally known therapeutic threshold for the treatment or alleviation of pain in a patient. In the context of the present invention the sub-analgesic amount of tramadol, or a salt thereof, will be considered a therapeutically effective anti-depressive amount. A “sub-analgesic” amount of tramadol, or a salt thereof, in accordance with the present invention may comprise <100 mg daily dose, e.g. 99 mg, or ≦90 mg daily dose, or ≦80 mg daily dose, or ≦75 mg daily dose, measured as tramadol hydrochloride.

The minimum dose for a “sub-analgesic” amount of tramadol, or a salt thereof, may be ≧10 mg daily dose, or ≧20 mg daily dose, or ≧30 mg daily dose, or ≧40 mg daily dose, or ≧50 mg daily dose, measured as tramadol hydrochloride.

Therefore, a preferred dose for a “sub-analgesic” amount of tramadol, or a salt thereof, may be from ≧10 mg to <100 mg daily dose, or 20 mg to 95 mg daily dose, or 30 mg to 90 mg daily dose, or 40 mg to 85 mg daily dose, or 50 mg to 80 mg daily dose, or 60 mg to 80 mg daily dose, or 65 mg to 75 mg daily dose, for example about 70 mg daily dose, measured as tramadol hydrochloride.

The use of a sub analgesic dose of tramadol, or a salt thereof, as hereinbefore described is advantageous in the treatment of a number of disorders associated with or defined under the broad heading of depression. Such disorders include, but shall not be limited to, major depression, chronic mild depression, manic depression (bipolar disorder), atypical depression, psychotic depression and dysthymia.

Thus, it will be understood that a large number of patients suffering from depression will be responsive to treatment with a sub-analgesic dose of tramadol, or a salt thereof, as hereinbefore defined. However, the use of a sub-analgesic dose of tramadol or a salt thereof is especially found to be useful in the patient group identified as “non-responders”, that is, the group of patients who show no or insufficient response to the use of SSRIs. As hereinbefore described, by the term insufficient response is generally meant, for example, a patient failing to achieve a 50% reduction in depressive symptoms, when measured according to the Hamilton Depression Rating Scale (HDRS).

It is understood that patients who are administered a conventional dose of tramadol for use as an analgesic, for example, ≧100 mg daily dose of tramadol, may experience some adverse effects, such as, constipation and in some cases diarrhoea; dizziness; drowsiness; increased sweating; loss of appetite; and/or nausea. Therefore the use of a sub-analgesic dose of tramadol, or a salt thereof, for the treatment, alleviation or prevention of depression, is advantageous in that, a patient may, in addition to receiving treatment for depression, they may, inter alia, experience fewer or less severe adverse effects, such as, but not limited to, those hereinbefore described, at least in part due to the lower dose of tramadol that is administered.

According to a further aspect of the invention there is provided the use of a sub-analgesic amount of tramadol, or a salt thereof, in the manufacture of a medicament for the treatment, alleviation or prevention of depression.

The invention further provides a pharmaceutical composition comprising a sub-analgesic amount of tramadol, or a salt thereof, in association with a pharmaceutically acceptable adjuvant, diluent or carrier. The pharmaceutical composition according to this aspect of the invention comprises a sub-analgesic amount of tramadol, or a salt thereof, in association with a pharmaceutically acceptable adjuvant, diluent or carrier, for use in the treatment, alleviation or prevention of depression.

The pharmaceutical composition of this aspect of the invention as hereinbefore described may contain sufficient of the tramadol, or a salt thereof, for a single daily sub-analgesic dose. Thus, the amount of the tramadol, or a salt thereof, present in the composition of the present invention may comprise <100 mg, e.g. 99 mg, or ≦90 mg, or ≦80 mg, or ≦75 mg, measured as tramadol hydrochloride, for use in the treatment, alleviation or prevention of depression. The minimum amount of tramadol, or a salt thereof, present in the composition may be ≧10 mg, or ≧20 mg, or ≧30 mg, or ≧40 mg, or ≧50 mg, measured as tramadol hydrochloride, for use in the treatment, alleviation or prevention of depression. Therefore, a preferred amount of tramadol, or a salt thereof, in the composition of the present invention may be from ≧10 mg to <100 mg, or 20 mg to 95 mg, or 30 mg to 90 mg, or 40 mg to 85 mg, or 50 mg to 80 mg, or 60 mg to 80 mg, or 65 mg to 75 mg, for example about 70 mg, measured as tramadol hydrochloride, for use in the treatment, alleviation or prevention of depression.

According to the FDA, pharmaceutical compositions are available, for example, in the form of tablets, comprising tramadol hydrochloride in an amount, 50 mg, 100 mg, 200 mg and 300 mg, for use as an analgesic.

An object of the present invention is, inter alia, to provide a daily dosage of tramadol, or a salt thereof, comprising <100 mg, e.g. 99 mg, of tramadol, measured as tramadol hydrochloride. Therefore, for example, for use with a twice daily (bd) or four times daily (qds) dosage regimen for the treatment or alleviation of depression, a pharmaceutical composition may comprise <50 mg of tramadol, or a salt thereof, measured as tramadol hydrochloride. Such a pharmaceutical composition is novel per se.

Therefore, according to a further aspect of the invention there is provided a pharmaceutical composition comprising from ≧10 mg to <50 mg tramadol, or a salt thereof, measured as tramadol hydrochloride, in association with a pharmaceutically acceptable adjuvant, diluent or carrier. Preferably, the composition according to this aspect of the invention is for use in the treatment, alleviation or prevention of depression.

The composition according to this aspect of the invention may comprise from ≧20 mg to <50 mg tramadol, or ≧30 mg to <50 mg tramadol, or ≧40 mg to <50 mg tramadol, or a salt thereof, measured as tramadol hydrochloride.

According to a yet further aspect of the invention there is provided a method of treatment of a patient suffering from depression, said method comprising the administration of a sub-analgesic amount of tramadol, or a salt thereof. Said sub-analgesic amount of tramadol, or a salt thereof, will be considered a therapeutically effective anti-depressive amount.

According to this aspect of the invention the method of treatment as hereinbefore described may comprise the administration of tramadol, or a salt thereof, in an amount of <100 mg daily dose, e.g. 99 mg, or ≦90 mg daily dose, or ≦80 mg daily dose, or ≦75 mg daily dose, measured as tramadol hydrochloride, for use in the treatment, alleviation or prevention of depression. The minimum amount of tramadol, or a salt thereof, administered according to this aspect of the invention may be ≧10 mg daily dose, or ≧20 mg daily dose, or ≧30 mg daily dose, or ≧40 mg daily dose, or ≧50 mg daily dose, measured as tramadol hydrochloride. Therefore, a preferred amount of tramadol, or a salt thereof, administered according to this aspect of the invention may be from ≧10 mg to <100 mg daily dose, or 20 mg to 95 mg daily dose, or 30 mg to 90 mg daily dose, or 40 mg to 85 mg daily dose, or 50 mg to 80 mg daily dose, or 60 mg to 80 mg daily dose, or 65 mg to 75 mg daily dose, for example about 70 mg daily dose, measured as tramadol hydrochloride.

The method of treatment according to this aspect of the invention especially comprises the administration a sub-analgesic dose of tramadol, or a salt thereof, to a patient that is in the patient group identified as “non-responders”, that is, the group of patients who fail to achieve a 50% reduction in depressive symptoms, when measured according to the Hamilton Depression Rating Scale (HDRS).

The dosing regimen may comprise once daily administration, or twice, three or four times daily.

The term “salt”, as used herein, shall refer to “pharmaceutically acceptable salts”, which are salts that retain the biological effectiveness and properties of tramadol and, which are not biologically or otherwise undesirable. Pharmaceutically acceptable acid addition salts can be formed with inorganic acids and organic acids, e.g., acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts. Inorganic acids from which salts can be derived include, for example, hydrochloric acid, hydrobromic acid, sulphuric acid, nitric acid, phosphoric acid, hexafluorophosphoric acid, and the like. A preferred salt of tramadol is the hydrochloride salt.

Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulphonic acid, ethanesulphonic acid, p-toluenesulphonic acid, salicylic acid, and the like. The pharmaceutically acceptable salts of tramadol can be prepared by conventional chemical methods known per se.

Reference to tramadol, or a salt thereof, shall include, inter alia, solvates. It may be convenient or desirable to prepare, purify, and/or handle a corresponding solvate of tramadol, which may be used in any one of the uses/methods described. The term solvate is used herein to refer to a complex of solute, such as a compound or salt of the tramadol and a solvent. If the solvent is water, the solvate may be termed a hydrate, for example a mono-hydrate, di-hydrate, tri-hydrate etc, depending on the number of water molecules present per molecule of substrate.

In the treatment, alleviation or prevention of depression as hereinbefore described, tramadol, or a salt thereof, may be administered alone or in combination with one or more other therapeutically active agents suitable for the treatment of depression. The use of combination partners may comprise separate administration of another therapeutically active agent suitable for the treatment of depression or may comprise a fixed combination, i.e., a single galenical composition comprising tramadol, or a salt thereof, and at least one combination partner. Such combination therapies may be prepared in a manner known per se and are those suitable for enteral, such as oral or rectal, and parenteral administration to mammals, including man.

Accordingly, and in another embodiment, the present invention provides a pharmaceutical composition sub-analgesic amount of tramadol, or a salt thereof, in combination with a second therapeutically active ingredient.

Said second therapeutically active ingredient will desirably be one which is efficacious in the treatment, alleviation or prevention of depression. Such, therapeutically active ingredients include but shall not be limited to, SSRI (other than tramadol), a tricyclic antidepressant (TCA), bupropion or a monoamine oxidase inhibitor (MAOI). In combination with a sub-analgesic amount of tramadol, or a salt thereof, the aforementioned second therapeutically active ingredient may be administered in lower than usual doses and therefore some of the undesirable adverse effects may be avoided or mitigated. Specific SSRIs which may be mentioned include, but shall not be limited to, one or more of citalopram, escitalopram, fluoxetine (Prozac®) paroxetine and sertraline. Specific TCAs which may be mentioned include, but shall not be limited to, one or more of amineptine, amitriptyline, butriptyline, clomipramine, demexiptiline, desipramine, dibenzepin, dimetacrine, dosulepin, doxepin, imipramine, iprindole, lofepramine, meltracen, metapramine, nitroxazepine, nortriptyline, noxiptiline, opipramol, pipofezine, propizepine, protriptyline, quinupramine, tianeptine and trimipramine. Specific MAOIs which may be mentioned include, but shall not be limited to, one or more of non-selective MAO-A/MAO-B inhibitors, such as, hydrazines: benmoxin, hydralazine, iproclozide, iproniazid, isocarboxazid, isoniazid, mebanazine, nialamide, octamoxin, phenelzine, pheniprazine, phenoxypropazine, pivalylbenzhydrazine, procarbazine and safrazine; non-hydrazines: caroxazone, echinopsidine, furazolidone, linezolid and tranylcypromine; selective MAO-A inhibitors, such as, brofaromine, metralindole, minaprine, moclobemide, pirlindole, toloxatone, amiflamine, bazinaprine, befloxatone, befol, cimoxatone, clorgyline, esuprone, sercloremine, tetrindole, thesputiaint and tyrima; selective MAO-B inhibitors, such as, lazabemide, pargyline, rasagiline, selegiline, D-deprenyl, ladostigil, milacemide and mofegiline.

Other suitable second therapeutically active ingredient which may be mentioned, include, but shall not be limited to, resveratrol, or acetaminophen, xorphanol, cinfenoac, furcloprofen, bismuth subsalicylate, enofelast, triflusal, ketorfanol, indriline, furofenac, cizolirtine, dacemazine, demelverine, fenethazine and/or derivatives thereof and combinations thereof.

Advantageously, in the use and or method of treatment of the present invention the tramadol, or a salt thereof, and/or a combination with a second therapeutically active ingredient may be administered enterally, e.g. orally, or intravenously.

Thus, in the use, method and/or composition of the invention the a sub-analgesic dose of tramadol, or a salt thereof, and/or a combination with a second therapeutically active ingredient may be put up as a tablet, capsule, dragee, suppository, suspension, solution, injection, e.g. intravenously, intramuscularly or intraperitoneally, implant, a topical, e.g. transdermal, preparation such as a gel, cream, ointment, aerosol or a polymer system, or an inhalation form, e.g. an aerosol or a powder formulation.

Compositions suitable for oral administration include tablets, capsules, dragees, liquid suspensions, solutions and syrups;

compositions suitable for topical administration to the skin include creams, e.g. oil-in-water emulsions, water-in-oil emulsions, ointments or gels;

examples of such adjuvants, diluents or carriers are:

for tablets and dragees—fillers, e.g. lactose, starch, microcrystalline cellulose, talc and stearic acid; lubricants/glidants, e.g. magnesium stearate and colloidal silicon dioxide; disintegrants, e.g. sodium starch glycolate and sodium carboxymethylcellulose;

for capsules—pregelatinised starch or lactose;

for oral or injectable solutions or enemas—water, glycols, alcohols, glycerine, vegetable oils;

for suppositories—natural or hardened oils or waxes.

It may be possible to administer a compound of the invention and/or derivatives and/or combinations thereof or any combined regime as described above, transdermally via, for example, a transdermal delivery device or a suitable vehicle or, e.g. in an ointment base, which may be incorporated into a patch for controlled delivery. Such devices are advantageous, as they may allow a prolonged period of treatment relative to, for example, an oral or intravenous medicament.

Examples of transdermal delivery devices may include, for example, a patch, dressing, bandage or plaster adapted to release a compound or substance through the skin of a patient. A person of skill in the art would be familiar with the materials and techniques which may be used to transdermally deliver a compound or substance and exemplary transdermal delivery devices are provided by GB2185187, U.S. Pat. No. 3,249,109, U.S. Pat. No. 3,598,122, U.S. Pat. No. 4,144,317, U.S. Pat. No. 4,262,003 and U.S. Pat. No. 4,307,717.

In a further embodiment, the methods and medicaments described herein may be used prophylactically as a means to prevent the development of depression. Medicaments and/or methods for prophylactic use may be administered or applied to any person at risk of developing depression.

For man the indicated total daily dosage may vary, but may be in the range of from 1 mg to 3,000 mg, preferably 5 mg to 500, which may be administered in divided doses from 1 to 6 times a day or in sustained release form.

The pharmaceutical composition of the invention may be in immediate release form or controlled release form. Therefore, according to a further aspect of the invention there is provided a controlled release pharmaceutical composition comprising a sub-analgesic amount of tramadol, or a salt thereof, in association with a pharmaceutically acceptable adjuvant, diluent or carrier, for use in the treatment, alleviation or prevention of depression.

Optionally, the pharmaceutical composition of this aspect of the invention may comprise an immediate release portion and a controlled release portion. When the composition of the present invention comprises a sub-analgesic dose of tramadol, or a salt thereof, as active ingredient, in combination with a second therapeutically active ingredient, one active ingredient may be in immediate release form and the other in controlled release form.

The present invention further provides a once daily oral pharmaceutical composition for controlled release of a sub-analgesic dose of tramadol, or a salt thereof, optionally in combination with a second therapeutically active ingredient, in which the composition, upon initial administration, provides an onset of antidepressive effect within 2 hours, which antidepressive effect continues for at least 24 hours after administration.

In accordance with another aspect of the present invention, there is provided a once daily oral pharmaceutical composition for controlled release of a sub-analgesic dose of tramadol, or a salt thereof, optionally in combination with a second therapeutically active ingredient, as hereinbefore described, wherein the composition, when ingested orally, provides a clinical effect over 24 hours which is at least as good as the clinical effect over 24 hours of two doses of a twice daily oral pharmaceutical composition for controlled release of a sub-analgesic dose of tramadol, or a salt thereof, taken 12 hours apart.

Although an advantage of the use of a sub-analgesic dose of tramadol, or a salt thereof, is that it minimises the risk of abuse, according to a further aspect of the invention there is provided an abuse resistant controlled release pharmaceutical composition comprising tramadol, or a salt thereof, in admixture with a suitable adjuvant, diluent or carrier.

The abuse resistant controlled release pharmaceutical composition as hereinbefore described is preferably a composition that is suitable for a once daily dosage regime of treatment.

Thus, the present invention provides an oral pharmaceutical composition and/or the use thereof for preventing or minimising the risk of abuse from either intentional or unintentional tampering.

The abuse resistant pharmaceutical composition and method of the present invention provides abuse deterrence and controlled release. It will be understood by the person skilled in the art that the abuse resistance and/or deterrence and controlled release may occur simultaneously, sequentially or separately.

The abuse resistant pharmaceutical composition may comprise one or more abuse resistant components selected from the group consisting of, hydrogenated vegetable oil; polyoxyethylene stearate (optionally including distearate); glycerol monostearate; poorly water soluble, high melting point wax, and mixtures thereof By the term “high melting point wax” we mean a wax with a melting point of from 45 to 100° C.

The abuse resistant pharmaceutical composition may also include one or more glyceryl fatty acid esters (including monoesters, diesters and triesters). Although it will be understood that a wide range of glyceryl fatty acid esters are available, examples of such esters include, but shall not be limited to, glyceryl behenate, glyceryl palmitostearate; macrogol glycerides, such as, stearoyl macrogolglycerides and lauroyl macrogolglyceride.

Examples of hydrogenated vegetable oils of the present invention include, but shall not be limited to, hydrogenated cottonseed oil, hydrogenated palm oil, hydrogenated soybean oil and hydrogenated palm kernel oil. Examples of polyoxyethylene stearates and distearates of the present invention include, but shall not be limited to, polyoxyl 2, 4, 6, 8, 12, 20, 30, 40, 50, 100 and 150 stearates, PEG-2 stearate, PEG-4 stearate, PEG 300 monostearate, PEG 600 monostearate, PEG-30 stearate, polyoxyethylene (30) stearate, polyoxyl 4, 8, 12, 32 and 150 distearates, PEG-4 distearate, PEG 400 distearate, PEG 600 distearate and PEG 1540 distearate. Examples of poorly water soluble, high melting point waxes of the present invention include, but shall not be limited to, animal waxes, insect waxes, vegetable waxes, mineral waxes, petroleum waxes, synthetic waxes, non-ionic emulsifying waxes, cetomacrogol emulsifying wax, anionic emulsifying wax, carnauba wax, caranda wax, microcrystalline wax, petroleum ceresin, microcrystalline petroleum wax, yellow wax (yellow beeswax), refined wax, white wax (bleached wax), cetyl esters wax, hydrogenated castor oil, lanolin alcohols, (e.g., cholesterol; lanolin; lanolin, hydrous; petrolatum and lanolin alcohols; mineral oils), anhydrous lanolin, refined wool fat, glyceryl palmitostearate and cetostearyl alcohol (e.g., cetearyl alcohol).

The abuse resistant composition may include a surfactant. Surfactants may be hydrophilic or hydrophobic, hydrophilic surfactants may be selected from the group consisting of non-ionic hydrophilic surfactants and anionic hydrophilic surfactants or the surfactant may have hydrophobic properties; and mixtures thereof. Examples of non-ionic hydrophilic surfactants include polyoxyethylene sorbitan esters, cremophores and poloxamers. Examples of anionic surfactants are sodium lauryl sarcosinate, docusate and pharmaceutically acceptable docusate salts.

The abuse resistant composition of the invention may optionally comprise other “auxiliary” materials, including:

Binders, such as, acacia, alginic acid and salts thereof, cellulose derivatives, methylcellulose, hydroxyethyl cellulose, hydroxypropyl cellulose, magnesium aluminium silicate, polyethylene glycol, gums, polysaccharide acids, bentonites, hydroxypropyl methylcellulose, gelatin, polyvinylpyrrolidone, polyvinylpyrrolidone/vinyl acetate copolymer, crospovidone, povidone, polymethacrylates, hydroxypropylmethylcellulose, hydroxypropylcellulose, starch, pregelatinised starch, ethylcellulose, tragacanth, dextrin, microcrystalline cellulose, sucrose, glucose, etc;

Disintegrants, such as, starches, pregelatinised corn starch, pregelatinised starch, celluloses, cross-linked carboxymethylcellulose, crospovidone, cross-linked polyvinylpyrrolidone, a calcium or a sodium alginate complex, clays, alginates, gums, or sodium starch glycolate, and any disintegration agents used in tablet preparations; Filling agents, such as, lactose, calcium carbonate, calcium phosphate, dibasic calcium phosphate, calcium sulphate, microcrystalline cellulose, cellulose powder, dextrose, dextrates, dextran, starches, pregelatinised starch, sucrose, xylitol, lactitol, mannitol, sorbitol, sodium chloride, polyethylene glycol, and the like; (iv) Stabilizers such as any antioxidation agents, reducing agents, buffers, or acids, sodium citrate, ascorbyl palmitate, propyl gallate, ascorbic acid, vitamin E, sodium bisulphite, butylhydroxyl toluene, BHA, acetylcysteine, monothioglycerol, phenyl-alpha-naphthylamine, lecithin, EDTA, etc.

Lubricants, such as, magnesium stearate, calcium hydroxide, talc, colloidal silicon dioxide, sodium stearyl fumarate, hydrogenated vegetable oil, stearic acid, glyceryl behenate, magnesium, calcium and sodium stearates, stearic acid, talc, waxes, boric acid, sodium benzoate, sodium acetate, sodium chloride, DL-leucine, polyethylene glycols, sodium oleate, sodium lauryl sulphate, etc.

Wetting agents, such as, oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulphate, etc.

Diluents, such as, lactose, starch, mannitol, sorbitol, dextrose, microcrystalline cellulose, dibasic calcium phosphate, sucrose-based diluents, confectioner's sugar, monobasic calcium sulphate monohydrate, calcium sulphate dihydrate, calcium lactate trihydrate, dextrates, inositol, hydrolyzed cereal solids, amylose, powdered cellulose, calcium carbonate, glycine, bentonite, etc.

Glidants (or anti-adherants), such as, talc, corn starch, DL-leucine, sodium lauryl sulphate, and magnesium, calcium, sodium stearates, etc.

Pharmaceutically acceptable carriers, such as, acacia, gelatin, colloidal silicon dioxide, calcium glycerophosphate, calcium lactate, maltodextrin, glycerin, magnesium silicate, sodium caseinate, soy lecithin, sodium chloride, tricalcium phosphate, dipotassium phosphate, sodium stearoyl lactylate, carrageenan, monoglyceride, diglyceride, pregelatinised starch, etc.

Other pharmaceutical excipients, such as, polymers, hydrogels, silicon dioxide, ion exchange resins, cellulose acetate butyrate, carbohydrate polymers, organic acids of carbohydrate polymers caprylic/capric triglyceride, isopropyl myristate, ethyl oleate, triethyl citrate, dimethyl phthalate, and benzyl benzoate.

The abuse resistant composition of the invention may further contain one or more pharmaceutically acceptable excipients which may play a role in the behaviour of the abuse resistant composition in the gastrointestinal tract.

The composition of the present invention may optionally include one or more other therapeutic agents in immediate or controlled release form; and optionally one or more excipients or auxiliary agents, such as glidants, lubricants, disintegrants, antistatic agents, solvents, channel forming agents, coating agents, flavourants, preservatives, bulking agents, polymers, etc. and inert carriers; wherein the dosage form provides for deterrence of abuse of the analgesic anti-depressant drug.

In particular, the dosage form may resist, deter or prevent crushing, shearing, grinding, chewing, dissolving, melting, needle aspiration, inhalation, insufflation or solvent extraction of the analgesic anti-depressant drug. Preferably the dosage for provides or assists in providing controlled release of the analgesic anti-depressant drug.

The administration of a once daily dosage may be advantageous because, inter alia, if a side-effect of dizziness is experienced, the consequences may be minimised by the administration of a once daily dosage to a patient at night time, i.e. before bed time.

The composition of the present invention may further optionally include one or more other therapeutic agents in immediate or controlled release form; and optionally one or more excipients or auxiliary agents, such as glidants, lubricants, disintegrants, antistatic agents, solvents, channel forming agents, coating agents, flavourants, preservatives, bulking agents, polymers, etc. and inert carriers; wherein the dosage form provides for deterrence of abuse of the analgesic anti-depressant drug.

In particular, the dosage form may resist, deter or prevent crushing, shearing, grinding, chewing, dissolving, melting, needle aspiration, inhalation, insufflation or solvent extraction of the tramadol, or a salt thereof. Preferably the dosage form provides or assists in providing controlled release of the sub-analgesic dose of tramadol, or a salt thereof.

The use as hereinbefore described especially comprises the use of a sub-analgesic dose of tramadol, or a salt thereof, in the manufacture of a medicament for the treatment of depression.

The controlled release composition is especially suitable for a once daily dosage regime of treatment.

As used herein, the term “salts” refers to salts that retain the biological effectiveness and properties of the therapeutically effective compounds described herein. Pharmaceutically acceptable acid addition salts can be formed with inorganic acids and organic acids, e.g., acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts. Inorganic acids from which salts can be derived include, for example, hydrochloric acid, hydrobromic acid, sulphuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulphonic acid, ethanesulphonic acid, p-toluenesulphonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminium, and the like. It may be convenient or desirable to prepare, purify, and/or handle a corresponding solvate of the compounds described herein, which may be used in any one of the uses/methods described. The term solvate is used herein to refer to a complex of solute, such as a compound or salt of the compound, and a solvent. If the solvent is water, the solvate may be termed a hydrate, for example a mono-hydrate, di-hydrate, tri-hydrate etc, depending on the number of water molecules present per molecule of substrate.

The term “controlled release” is defined for purposes of the present invention as a method of oral drug delivery where the rate of release of the active pharmaceutical ingredient from the formulation is not solely dependent on the concentration of active pharmaceutical ingredient remaining in the formulation and/or the solubility of the active pharmaceutical ingredient in the medium surrounding the formulation, and where the time course and/or location of release of an active ingredient from a pharmaceutical formulation are chosen to accomplish therapeutic or convenience objectives not offered by conventional dosage forms.

The dosage form of the invention may include both an immediate release and extended or controlled release component.

In a further aspect of the invention we provide a novel method for reducing one or more of:
the peak concentration (C_{maχ}) an anti-depressant;
the early post-dose partial area under the plasma concentration time curve an anti-depressant;
the early post-dose average plasma concentration time (Cave) an anti-depressant;
the intensity of an anti-depressant toxicity upon tampering; and
the intensity or frequency of one or more signs and symptoms of anti-depressant toxicity, including nausea, vomiting, somnolence, stupor, coma, respiratory depression, apnoea, respiratory arrest, circulatory depression, bradycardia, hypotension, shock and skeletal muscle flaccidity.

The orally administered pharmaceutical composition may generally be a tablet form. However, it will be understood by the person skilled in the art that the dosage form may be, for example, a capsule in which the sub-analgesic dose of tramadol, or a salt thereof, may be present in the form of controlled release granules or the like. Therefore, in the following description reference to the structure of a controlled release tablet will be understood by the person skilled in the art to be applicable to for example granules which may be made up in capsule form.

Thus, the controlled release composition of the invention may comprise a tablet or granule comprising a core e.g. a controlled release core and a coating, optionally a controlled release coating.

The core of a tablet or granule of the invention includes a sub-analgesic dose of tramadol, or a salt thereof, and a matrix, these components associated in such a way that release of the sub-analgesic dose of tramadol, or a salt thereof, from the matrix is controlled. In a specific embodiment, the matrix of the core is a cross-linked high amylose starch which is described most recently in U.S. Pat. No. 6,607,748.

Preferably, the core is formed by admixing the ingredients (in granular or powder form) and then compressing the mixture to form the core over which the coat is subsequently formed The weight of the core can be any percentage of the weight of the total composition between 10% and 80%. The preferred percentage depends, upon other things, the total dosage of the sub-analgesic dose of tramadol, or a salt thereof. In a particular embodiment described further below, a tablet contains a sub-analgesic dose of tramadol, or a salt thereof, as hereinbefore described and the core is about 26% of the total weight of the tablet. In another embodiment, the core makes up about 33% of the total weight of the tablet.

The release from the extended or controlled release dosage composition of an active compound as hereinbefore described located in the core is slower than the release of an active compound as hereinbefore described located in the matrix of the coat. A preferred matrix of the core is cross-linked high amylose starch, described in U.S. Pat. No. 6,607,748. In particular embodiments, the matrix makes up between about 10% and about 90% by weight of the core i.e., the ratio of the matrix of the core to the active ingredient of the core (w/w) is between about 0.1 and about 10, or between about 0.2 and about 9, or between about 0.2 and about 8, or between about 0.3 and about 7, or between about 0.4 and about 6, or between about 0.5 and about 5, or between about 0.6 and about 4, or between about 0.7 and about 4 or between about 1 and about 4, or between about 1 and about 3 and about 1.5 and about 2.5. In one particular embodiment, the core totals about 90 mg, of which about 44 mg is cross-linked high amylose starch, and 45 mg is active compound as hereinbefore described. The cross-linked high amylose starch thus makes up about 49 weight percent of the core.

The core composition of the extended or controlled release dosage forms of the present invention may optionally include a pharmaceutically acceptable carrier or vehicle. Such carriers or vehicles are known to those skilled in the art and are found, for example, in Remington's Pharmaceutical Sciences, 14^{th} Ed. (1970). Examples of such carriers or vehicles include lactose, starch, dicalcium phosphate, calcium sulphate, kaolin, mannitol and powdered sugar. Additionally, when required, suitable binders, lubricants, and disintegrating agents can be included. If desired, dyes, as well as sweetening or flavouring agents can be included.

The core composition of the extended or controlled release dosage forms of the present invention may optionally include accessory ingredients including, but not limited to dispersing agents such as microcrystalline cellulose, starch, cross-linked starch, cross-linked poly(vinyl pyrrolidone), and sodium carboxymethyl cellulose; flavouring agents; colouring agents; binders; preservatives; surfactants and the like.

The core can, optionally, also include one or more suitable binders known to one of ordinary skilled in the art.

Suitable forms of microcrystalline cellulose, for example; MCC-PH101, MCC-102, MCC-105, etc.

Suitable lubricants, such as those known to the skilled person, may also be included for example, magnesium stearate, vegetable oil, talc, sodium-stearyl fumarate, calcium stearate, stearic acid, etc.

Suitable glidants, known in the art, may also be included. Examples of such glidants include, but are not limited to talc, colloidal silicon dioxide, etc.

The sub-analgesic dose of tramadol, or a salt thereof, may be present at levels ranging from about 1 to about 90% w/w of the total weight of the core, preferably from about 10 to about 70% w/w of the total composition of the core, more preferably from about 20 to about 60% w/w of the total composition of the core, and probably most often between about 30 to about 50% w/w of the total composition of the core.

Of course, the total amount of all components is 100% w/w, and those of ordinary skill in the art can vary the amounts within the stated ranges to achieve useful compositions.

The coat of the dosage form includes a physical mixture of polyvinyl acetate and polyvinylpyrrolidone and the active pharmaceutical ingredient(s) of the coat. The coat can also include a cross-linked high amylose starch and optionally other components. In a preferred embodiment, the coat is formed by dry compression. The weight of the coat can be any percentage of the weight of the total composition between about 10% and about 90%, but is preferably in the higher part of this range.

The coat thus usually makes up between about 20% to about 90%, (w/w) of a tablet of the invention, or about 35% to about 85%, or about 40% to about 85%, or about 45% to about 85%, or about 45% to about 90%, or about 60% to about 75%, or about or about 65% or about 70% or about 75%. The coat may include an optional binding agent.

The weight percentage of the polyvinyl acetate/polyvinylpyrrolidone mixture in the coat can be anywhere within a wide range of values Depending on the solubility in water of the active ingredient in the coat, the amount of the polyvinyl acetate/polyvinylpyrrolidone mixture in the coat can be adjusted. US Patent application No. 2001/0038852 describes ways in which such adjustments can be made. For example, for active ingredients that are soluble to extremely soluble in water, polyvinyl acetate/polyvinylpyrrolidone mixture can be about 20 to about 80% w/w of the coat, preferably about 30 to about 65% w/w, or about 40 to about 55% w/w.

The weight ratio of polyvinyl acetate to polyvinylpyrrolidone in the polyvinyl acetate/polyvinylpyrrolidone mixture can be a wide range of values. Preferably, such ratio is between about 6:4 and 9:1; more likely between about 7:3 and 6 DEG 1, even more preferably about 8:2.

The molecular weight of the polyvinyl acetate component in the polyvinyl acetate/polyvinylpyrrolidone mixture can be a wide range of values. Preferably, the average molecular weight of the polyvinyl acetate is about 100 to about 10,000,000; or about 1,000 to about 1,000,000; or about 10,000 to about 1,000,000; or about 100,000 to about 1,000,000; or about 450,000.

The molecular weight of the polyvinylpyrrolidone component in the polyvinyl acetate/polyvinylpyrrolidone mixture can be a wide range of values. The average molecular weight of the polyvinylpyrrolidone can be from about 100 to about 10,000,000; or about 1,000 to about 1,000,000; or about 5,000 to about 500,000; or about 10,000 to about 100,000; or about 50,000.

The polyvinyl acetate and polyvinylpyrrolidone mixture can be prepared by a variety of processes including simply mixing powders of polyvinylpyrrolidone and polyvinyl acetate. In a preferred embodiment, such mixture is spray dried powder of a colloidal dispersion of polyvinyl acetate and polyvinylpyrrolidone solution. Optionally, sodium lauryl sulphate is used as a stabilizer in order to prevent agglomeration during spray drying process and/or colloidal silica is used to improve the flow properties of the polyvinyl acetate/polyvinylpyrrolidone mixture. Optionally, polyvinyl acetate and polyvinylpyrrolidone can be formed in a random or a block copolymer.

Suitable binding agents for the present invention include, but are not limited to, plant extracts, gums, synthetic or natural polysaccharides, polypeptides, alginates, synthetic polymers, or a mixture thereof.

Suitable plant extracts to be used as gelling agents include, but are not limited to, agar, ispaghula, psyllium, cydonia, ceratonia or a mixture thereof.

Suitable gums to be used as gelling agents include, but are not limited to, xanthan gum, guar gum, acacia gum, ghatti gum, karaya gum, tragacanth gum or a mixture thereof.

Suitable synthetics or natural hydrophilic polysaccharides to be used as gelling agents include, but are not limited to, hydroxyalkylcelluloses, cellulose ethers, cellulose esters, nitrocelluloses, dextrin, agar, carrageenan, pectin, furcellaran, starch or starch derivatives, cross-linked high amylose starch, or a mixture thereof.

Suitable polypeptides to be used as gelling agents include, but are not limited to, gelatin, collagen, polygeline or a mixture thereof.

Suitable alginates to be used as gelling agents include, but are not limited to, alginic acid, propylene glycol alginate, sodium alginate or a mixture thereof.

Suitable synthetic polymers to be used as gelling agents include, but are not limited to, carboxyvinyl polymer, polyvinyl alcohol, polyvinyl pyrrolidone, polyethylene oxide, polyethylene glycols, copolymers of ethylene oxide and propylene oxide and their copolymers or a mixture thereof.

In a preferred embodiment, the gelling agent is a gum such as xanthan gum, guar gum, acacia gum, ghatti gum, karaya gum, tragacanth gum or a mixture thereof, PEO 7,000,000 and HPMC K100 M.

In a most preferred embodiment, the gelling agent is xanthan gum.

The tablet or capsule composition of the present invention can be administered through, but not limited to, a number of routes such as oral, sublingual, and rectal. The preferred route of administration of the compositions of the present invention is oral.

Compositions of the present invention that are suitable for oral administration may be presented as discrete units such as tablets or granules. Preferably, the compositions of the present invention are presented in a tablet form. Such tablets may be conventionally formed by compression or moulding. Compressed tablets may be prepared by compressing in a suitable machine the mixture of one or more components described above. Moulded tablets may be made by moulding in a suitable machine the above components, which can be optionally moistened with an inert liquid diluent. The tablets may optionally be coated and/or have other identifying indicia visible to the consumer. A tablet can also be in a variety of forms, e.g., uncoated, dry coated, or film coated, etc. A tablet can also be in a variety of shapes (e.g. oval, sphere, etc.) and sizes. A comprehensive discussion of tablets can be found in references, such as, The Theory and Practice of Industrial Pharmacy by Lachman et al., 3<rd>Ed. (Lea & Febiger, 1986).

The sub-analgesic dose of tramadol, or a salt thereof, as active agent of the composition exhibits the following in vitro dissolution profile when measured with a USP Type I apparatus in 50 mM phosphate, pH 6.8, and stirring between 50 and 150 rpm.

An average rate of between 10% and 30% per hour of the sub-analgesic dose of tramadol, or a salt thereof, as active agent may be released between 0 and 2 hours when tested in vitro using a USP Type I apparatus in 50 mM phosphate, pH 6.8, and stirring between 50 and 150 rpm; or between 10% and 40% of the sub-analgesic dose of tramadol, or a salt thereof, as active agent may be released from the formulation between 0 and about 2 hours of measurement, between about 30% and 60% of the sub-analgesic dose of tramadol, or a salt thereof, as active agent may be released from the formulation between 2 and about 7 hours of the measurement, between about 50% and 80% of the sub-analgesic dose of tramadol, or a salt thereof, as active agent may be released from the formulation between 7 and about 12 hours of measurement, and between about 80% and 100% of the sub-analgesic dose of tramadol, or a salt thereof, as active agent may be released from the formulation after about 20 hours of measurement; or more preferably between 15% and 35% of the sub-analgesic dose of tramadol, or a salt thereof, as active agent may be released from the formulation between at 2 hours of measurement, between about 40% and 60% of the sub-analgesic dose of tramadol, or a salt thereof, as active agent may be released from the formulation between at 7 hours of the measurement, between about 60% and 80% of the sub-analgesic dose of tramadol, or a salt thereof, as active agent may be released from the formulation at 12 hours of measurement, and between about 85% and 100% of the sub-analgesic dose of tramadol, or a salt thereof, as active agent may be released from the formulation after about 20 hours of measurement, or between 20% and 40% of the sub-analgesic dose of tramadol, or a salt thereof, as active agent may be released from the formulation between at 2 hours of measurement, between about 40% and 60% of the sub-analgesic dose of tramadol, or a salt thereof, as active agent may be released from the formulation between at 7 hours of the measurement, between about 60% and 80% of the sub-analgesic dose of tramadol, or a salt thereof, as active agent may be released from the formulation at 12 hours of measurement, and between about 85% and 100% of the sub-analgesic dose of tramadol, or a salt thereof, as active agent may be released from the formulation after about 20 hours of measurement.

Alternatively, when the dosage form of the invention is an abuse resistant dosage form it may provide at least 60% of the steady state concentration of the sub-analgesic dose of tramadol, or a salt thereof, after administration of one dose at its intended dosing frequency, preferably at least about 62.5%, or at least about 65%, or at least about 67.5%, or at least about 70%, or at least about 72.5%,or at least about 75%, or at least about 77.5%, or at least about 80%, or at least about 82.5%,or at least about 85%, or at least about 87.5%, or at least about 90%, or at least about 92.5%, or at least about 95% or at least 98% of the steady state therapeutic concentration of the sub-analgesic dose of tramadol, or a salt thereof, after administration of one dose at its intended dosing frequency.

The amount of abuse resistant component(s) in the composition of the invention may be from about 1 mg to 1500 mg. In a preferred embodiment, the amount of anti-abuse components in the claimed composition may be from about 10 mg to 800 mg. In a most preferred embodiment, the amount of anti-abuse components in the claimed composition may be about 50 mg to 600 mg.

The ratio of the sub-analgesic dose of tramadol, or a salt thereof, to the anti-abuse components may be from about 1:10,000 to about 10,000:1 w/w, preferably from about 1:1000 to about 1000:1 w/w, more preferably from 1:250 to 250:1 w/w.

All oral pharmaceutical dosage forms of the invention are contemplated, including oral suspensions, tablets, capsules, lozenges, effervescent tablets, effervescent powders, powders, solutions, powders for reconstitution, transmucosal films, buccal products, oral mucoretentive products, oral gastroretentive tablets and capsules, orally disintegrating tablets, fast dissolving tablets, fast dispersing tablets, fast disintegrating dosage forms, administered as immediate release, delayed release, modified release, enteric coated, sustained release, controlled release, pulsatile release and extended release dosage form.

As used herein, “controlled release” is interchangeable with “extended release”, “sustained release”, “modified release”, “delayed release” and the like. Such products provide a longer duration of action than conventional immediate release formulations of the same drugs and are usually administered every 24 hours.

Controlled release dosage forms of the present invention release the analgesic-antidepressant from the oral dosage form at slower rate than immediate release formulations. The controlled release dosage form may release the sub-analgesic dose of tramadol, or a salt thereof, at such a rate that blood (e.g., plasma) concentrations (levels) or therapeutic effects are maintained within the therapeutic range (above the minimum effective therapeutic concentration) but below toxic levels for intended duration, e.g. over a period of from 1 to 24 hours or more,

In a further aspect of the invention there is provided a method and/or pharmaceutical composition to simultaneously achieve controlled release and abuse deterrence, without the use of aversive agents.

The abuse resistance may include, for example, resistance to significant changes in oral bioavailability due to changes in food intake.

Controlled release formulations of abusable drugs are often used due, inter alia, to the large amount of active ingredient per dosage form, a 24 hour supply. Tampering with controlled release formulations will generally rapidly deliver a massive dose and produce profound pharmacologic effects. Abusable drugs may be administered by a variety of routes, such as, parenteral (e.g., intravenous injection, where the drug may be crushed and extracted or melted and the contents of a dosage unit then injected); intranasal (e.g., snorting, where the drug is inhaled as powdered dosage unit). The most common method of abuse with antidepressants is oral ingestion of the crushed drug, for example, where the drug is chewed to increase the surface area and permit rapid release of antidepressant active ingredient.

All of these strategies are intended to more efficiently get the abusable drug into the patient, both in terms of total amount of drug, peak concentration of drug and time to peak concentration of drug.

It is necessary to be able to measure resistance or deterrence of the dosage form to the likely abuse. Thus, provided herein are exemplary in vitro tests, such as,

Extraction with Alcohol on Whole Dosage Unit

Extraction with Alcohol on a Crushed or Cut Dosage Unit

Extraction into Water

Freeze and Crush

Taste of Base Excipient Mix (organoleptic test)

Extraction into Acid

Application of Heat (melting temperature >50° C. or 55° C.

In one embodiment of the present invention the dosage form comprises subunits (a) and (b) which are present as for example, micro tablets, microcapsules, micro pellets, granules, spheroids, beads or pellets. Desirably the same form, i.e. shape, is selected for both subunit (a) and subunit (b), such that it is not possible to separate subunits (a) from (b) by mechanical selection. The multiparticulate forms may be of a size in the range from 0.1 to 3 mm, e.g. from 0.5 to 2 mm in size e.g. length or diameter.

The subunits (a) and (b) may be packaged in a capsule, suspended in a liquid or a gel or be press-moulded to form a tablet, wherein the final formulation in each case proceeds in such a manner that the subunits (a) and (b) are also retained in the resultant dosage form.

The subunits (a) and (b) may optionally be of identical shape so that they are not visually distinguishable from one another. This may be advantageous so that the abuser cannot separate one another by simple sorting. This may, for example, be achieved by the application of identical coatings which, apart from this disguising function, may also incorporate further functions, such as, for example, delayed release of one or more active ingredients or provision of a finish resistant to gastric juices on the particular subunits.

In a further aspect of this embodiment the respective subunits (a) and (b) may be arranged in layers relative to one another.

A variety of materials may be used, examples shall include, but shall not be limited to, alkylcelluloses hydroxyalkylcelluloses, glucans, scleroglucans, mannans, xanthans, copolymers of poly[bis(p-carboxyphenoxy-)propane and sebacic acid], e.g. molar ratio of 20:80, carboxymethylcelluloses, cellulose ethers, cellulose esters, nitrocelluloses, polymers based on acrylic or methacrylic acid and esters thereof, polyamides, polycarbonates, polyalkylenes, polyalkylene glycols, polyalkylene oxides, polyalkylene terephthalates, polyvinyl alcohols, polyvinyl ethers, polyvinyl esters, halogenated polyvinyls, polyglycolides, polysiloxanes and polyurethanes; and copolymers thereof.

Suitable materials may be selected from the group consisting of methylcellulose, ethylcellulose, hydroxypropylcellulose, hydroxypropylmethyl cellulose, hydroxybutylmethylcellulose, cellulose acetate, cellulose propionate (of low, medium or high molecular weight), cellulose acetate propionate, cellulose acetate butyrate, cellulose acetate phthalate, carboxymethylcellulose, cellulose triacetate, sodium cellulose sulphate, polymethyl methacrylate, polyethyl methacrylate, polybutyl methacrylate, polyisobutyl methacrylate, polyhexyl methacrylate, polyisodecyl methacrylate, polylauryl methacrylate, polyphenyl methacrylate, polymethyl acrylate, polyisopropyl acrylate, polyisobutyl acrylate, polyoctatdecyl acrylate, polyethylene, low density polyethylene, high density polyethylene, polypropylene, polyethylene glycol, polyethylene oxide, polyethylene terephthalate, polyvinyl alcohol, polyvinyl isobutyl ether, polyvinyl acetate and polyvinyl chloride.

Further suitable copolymers may comprise copolymers of butyl methacrylate and isobutyl methacrylate, copolymers of methyl vinyl ether and maleic acid of high molecular weight, copolymers of methyl vinyl ether and maleic acid monoethyl ester, copolymers of methyl vinyl ether and maleic anhydride and copolymers of vinyl alcohol and vinyl acetate.

A barrier layer may comprise one or more suitable biodegradable materials, such as, starch-filled polycaprolactone, aliphatic polyesteramides, aliphatic and aromatic polyester urethanes, polyhydroxyalkanoates, such as, polyhydroxybutyrates, polyhydroxyvalerates, casein and polylactides.

Furthermore, the aforementioned materials may optionally be blended with further conventional auxiliary substances known to those skilled in the art, for example, those selected from, but not limited to, glyceryl monostearate, semi-synthetic triglyceride derivatives, semi-synthetic glycerides, hydrogenated castor oil, glyceryl palmitostearate, glyceryl behenate, polyvinylpyrrolidone, gelatine, magnesium stearate, stearic acid, sodium stearate, talcum, sodium benzoate, boric acid and colloidal silica, fatty acids, substituted triglycerides, glycerides, polyoxyalkylene glycols and derivatives thereof.

In a further embodiment of this aspect of the invention the dosage form may comprise a separation layer (c). The separation layer may comprise substantially the same material as the barrier layer. The thickness of the separation layer may vary so as to achieve the desired release of the active ingredient from the barrier layer.

The dosage form according to this aspect of the invention, e.g. for oral administration, is particularly suitable for preventing oral, nasal and/or parenteral abuse of such active ingredients.

If the dosage form according to the invention is intended for oral administration, it may also desirably comprise a coating which is resistant to gastric juices and, for example, dissolves as a function of the pH value of the release environment. By means of this coating, it may be possible to ensure that the dosage form according to the invention passes through the stomach undissolved and the active ingredient is only released in the intestines of a patient. A coating which is resistant to gastric juices may dissolve at a pH of between 5 and 7.5.

In an alternative aspect of the invention the dosage form is an oral dosage form comprising the sub-analgesic dose of tramadol, or a salt thereof, e.g. as an agonist, and an antagonist, wherein the antagonist is present in a substantially non-releasable form (i.e., “sequestered”). Thus, the dosage form may contain an orally therapeutically effective amount of the sub-analgesic dose of tramadol, or a salt thereof, agonist, the dosage form providing a desired therapeutic effect. Because the antagonist is present in a substantially non-releasable form, it does not substantially block the therapeutic effect of the sub-analgesic dose of tramadol, or a salt thereof, agonist when the dosage form is orally administered intact.

In further embodiment in this aspect of the invention, the oral dosage form may be directed to an oral dosage form comprising (i) a sub-analgesic dose of tramadol, or a salt thereof, agonist in releasable form and (ii) a sequestered antagonist which is substantially not released when the dosage form is administered intact, such that the ratio of the amount of antagonist released from the dosage form after tampering to the amount of the antagonist released from the intact dosage form is about 4:1 or greater, based on the in vitro dissolution at 1 hour of the dosage form in 900 ml of Simulated Gastric Fluid using a USP Type II (paddle) apparatus at 75 rpm at 37° C. wherein the agonist and antagonist are interdispersed and are not isolated from each other in two distinct layers.

In another embodiment, the invention comprises an oral dosage form comprising (i) a sub-analgesic dose of tramadol, or a salt thereof, as agonist in releasable form and (ii) a sequestered antagonist which is substantially not released when the dosage form is administered intact, such that the ratio of the amount of antagonist released from the dosage form after tampering to the amount of the antagonist released from the intact dosage form is about 4:1 or greater, based on the in-vitro dissolution at 1 hour of the dosage form in 900 ml of Simulated Gastric Fluid using a USP Type II (paddle) apparatus at 75 rpm at 37° C., wherein the antagonist is in the form of multiparticulates individually coated with a sequestering material which substantially prevents release of the antagonist.

In certain embodiments of the invention, the release for the antagonist component of the formulation may be expressed in terms of a ratio of the release achieved after tampering, e.g., by crushing or chewing, relative to the amount released from the intact formulation.

In a further embodiment of the present invention, an antagonist in a substantially non-releasable form may be prepared by combining the antagonist with a pharmaceutically acceptable hydrophobic material. Thus, for example, antagonist particles may be coated with a coating that substantially prevents the release of the antagonist, the coating comprising the hydrophobic materials. Another example is an antagonist that is dispersed in a matrix that renders the antagonist to be substantially non-releasable, the matrix comprising the hydrophobic materials. In certain embodiments, the pharmaceutical acceptable hydrophobic material may comprise a cellulose polymer selected from the group consisting of ethylcellulose, cellulose acetate, cellulose propionate (lower, medium or higher molecular weight), cellulose acetate propionate, cellulose acetate butyrate, cellulose acetate phthalate and cellulose triacetate. Alternatively, the hydrophobic material may comprise one or more of polylactic acid, polyglycolic acid or a co-polymer of the polylactic and polyglycolic acid.

In a further embodiment the hydrophobic material may comprise a cellulose polymer selected from the group consisting of cellulose ether, cellulose ester, cellulose ester ether, and cellulose. Additional cellulose polymers useful for preparing an antagonist in a substantially non-releasable form according to this aspect of the invention may include acetaldehyde dimethyl cellulose acetate, cellulose acetate ethylcarbamate, cellulose acetate methylcarbamate, and cellulose acetate dimethylaminocellulose acetate.

An acrylic polymer useful for preparation of the antagonist in a substantially non-releasable form may include an acrylic resin comprising copolymers synthesized from acrylic and methacrylic acid esters (e.g., the copolymer of acrylic acid lower alkyl ester and methacrylic acid lower alkyl ester) containing about 0.02 to 0.03 mole of a tri (lower alkyl) ammonium group per mole of the acrylic and methacrylic monomers used. An example of a suitable acrylic resin is Eudragit® RS, which is a water insoluble copolymer of ethyl acrylate, methyl methacrylate and trimethylammoniummethyl methacrylate chloride in which the molar ratio of trimethylammoniumethyl methacrylate chloride to the remaining components (ethyl acrylate and methyl methacrylate) is 1:40. Acrylic resins such as Eudragit®RS may be used in the form of an aqueous suspension.

In certain embodiments of this aspect of the invention, the acrylic polymer may be selected from the group consisting of acrylic acid and methacrylic acid copolymers, methyl methacrylate copolymers, ethoxyethyl methacrylates, cyanoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), and glycidyl methacrylate co-polymers thereof.

When the antagonist in a substantially non-releasable form comprises antagonist particles coated with a coating that renders the antagonist substantially non-releasable, and when a cellulose polymer or an acrylic polymer is used for preparation of the coating composition, suitable plasticisers, e.g., acetyl triethyl citrate and/or acetyl tributyl citrate may also be admixed with the polymer. The coating may also contain additives well known to the person skilled in the art, such as, colouring agents, talc and/or magnesium stearate, etc.

The coating composition may be applied onto the antagonist particles by spraying it onto the particles using any suitable spray equipment known in the part. For example, a Wuster fluidised-bed system may be used in which an air jet, injected from underneath, fluidizes the coated material and effects drying while the insoluble polymer coating is sprayed on. The thickness of the coating will depend on the characteristics of the particular coating composition being used. However, it is well within the ability of one skilled in the art to determine by routine experimentation the optimum thickness of a particular coating required for a particular dosage form of the present invention.

The pharmaceutically acceptable hydrophobic material useful for preparing an antagonist in a substantially non-releasable form includes a biodegradable polymer comprising a poly(lactic/glycolic acid) (“PLGA”), a polylactide, a polyglycolide, a polyanhydride, a polyorthoester, polycaprolactones, polyphosphazenes, polysaccharides, proteinaceous polymers, polyesters, polydioxanone, polygluconate, polylactic-acid-polyethylene oxide copolymers, poly(hydroxybutyrate), polyphospho ester or mixtures or blends thereof.

In a yet further alternative aspect of the present invention the dosage form may comprise a co-extruded pharmaceutical composition including a sub-analgesic dose of tramadol, or a salt thereof, as an active agent and an adverse agent (antagonist). Thus, the dosage form in accordance with this aspect of the present invention may include an oral dosage form, including but not limited to, capsules or tablets, rectal suppositories and vaginal suppositories. The dosage form may comprise a co-extruded composition, including but not limited to one or more particles such as melt-extruded multiparticulates made by a process comprising co-extrusion.

In one embodiment of this aspect of the present invention, a co-extruded dosage form includes a core comprising an adverse agent (antagonist), and one or more shell layers or components comprising a sub-analgesic dose of tramadol, or a salt thereof, as an active agent. In this embodiment, the shell layers or components at least partially surround the core, and preferably, surround a majority of the core and most preferably the whole of the core. The dosage form is made by a process which comprises co-extrusion of the core and the shell.

In yet further embodiment, the invention relates to a co-extruded dosage form including a core, a sheath comprising one or more sheath layers or components, and a shell comprising one or more shell layers or components. The dosage form may be made by co-extrusion of the core, the sheath and the shell. In this embodiment, the core may comprise an adverse agent (antagonist), the sheath may comprise a hydrophobic material and the shell may comprise an active agent at least partially surrounding the sheath.

In one embodiment of this aspect of the invention the shell may comprise a controlled release form of a sub-analgesic dose of tramadol, or a salt thereof, as active agent. Also, in this embodiment, the sheath component may contribute to delaying and/or reducing the in vivo release of adverse agent (antagonist) contained in the core.

This aspect of the present invention may comprise a method of making a tamper-resistant dosage form comprising a) forming a multilayer extrudate by co-extruding a core comprising an adverse agent (antagonist) and a shell comprising a sub-analgesic dose of tramadol, or a salt thereof, as an active agent (agonist) which may at least partially surround the sheath; and b) rendering the multilayer extrudate to form at least one particle.

In this embodiment the present invention may include a method of making a tamper-resistant dosage form comprising a) forming a multilayer extrudate by co-extruding a core comprising an adverse agent (antagonist) and a hydrophobic material; a sheath comprising a hydrophobic material which at least partially surrounds the core; and a shell comprising a sub-analgesic dose of tramadol, or a salt thereof, as an active agent (agonist) and a hydrophobic material which at least partially surrounds the sheath; b) using a rolling punch to form one more particles from the multilayer extrudate; and c) incorporating one or more particles into a dosage form.

The particles or tablets of the invention may further comprise pharmaceutically acceptable hydrophobic coating materials; excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g. potato starch or sodium starch glycolate); wetting agents (e.g., sodium lauryl sulphate); and other additives or excipients or as is well-known in the art. Furthermore, the particles or tablets may be coated by methods well-known in the art provided such coating does not interfere with the intended use. Examples of coating processes are spray coating and dip coating, etc.

In certain embodiments the present invention, the adverse agent (antagonist), which may be sequestered, can be present in the core or in an inner layer of a co-extruded, multi-layer particle. The adverse agent-containing core may include a hydrophobic matrix material. Hydrophobic matrix materials useful in the present invention include, but shall not be limited to, those that are known in the art to be insoluble or to have a low solubility in the gastrointestinal tract. Such materials include, but are not limited to, a hydrophobic material, such as, acrylic and methacrylic acid polymers and copolymers, and alkylcelluloses. The matrix may also include additional hydrophobic materials such as zein, shellac, hydrogenated castor oil, hydrogenated vegetable oil or mixtures thereof. Although generally insoluble, such hydrophobic materials will degrade over time, thereby eventually releasing at least a portion of the adverse agent. The rate of release may be controlled by, for example, altering the content of the hydrophobic matrix material in the adverse agent core in order to alter the in vivo release of the adverse agent.

The hydrophobic matrix material may include acrylic polymers. Examples of suitable acrylic polymers include, but shall not be limited to acrylic acid and methacrylic acid copolymers, methyl methacrylate copolymers, ethoxyethyl methacrylates, cyanoethyl methacrylates, aminoalkyl methacrylate copolymer, poly(acrylic acid), poly(methacrylic acid), methacrylic acid alkylamide copolymers, poly(methyl methacrylate), polymethacrylate, poly(methyl methacrylate) copolymer, poly(methacrylic acid) (anhydride), methyl methacrylate, polyacrylamide, aminoalkyl methacrylate copolymer, poly(methacrylic acid anhydride), and glycidyl methacrylate copolymers. Additional examples of suitable acrylic polymers include, but are not limited to, acrylic resins including copolymers synthesized from acrylic and methacrylic acid esters (e.g. the copolymer of acrylic acid lower alkyl ester and methacrylic acid lower alkyl ester) containing, for example, about 0.02 to 0.03 moles of a tri (lower alkyl) ammonium group per mole of acrylic and methacrylic monomer.

The acrylic polymer can comprise one or more ammonium methacrylate copolymers. Ammonium methacrylate copolymers are well known in the art, and are fully polymerised copolymers of acrylic and methacrylic acid esters with a generally low content of quaternary ammonium groups. In order to obtain a desirable dissolution profile for a given therapeutic agent, it might be necessary to incorporate two or more ammonium methacrylate copolymers having differing physical properties. For example, it is known that by changing the molar ratio of the quaternary ammonium groups to neutral methacrylic esters, the permeability properties of the resultant coating can be modified. The ordinary person skilled in the art will readily be able to combine monomers to provide a copolymer that releases the therapeutic agent at the desired release rate. Co-polymers of acrylate and methacrylate having a quaternary ammonium group functionality are commercially available as Eudragit® RS. In one embodiment the hydrophobic matrix material may include a water insoluble cellulose polymer. The cellulose polymer may be a cellulose ether, a cellulose ester, or a cellulose ester ether. Preferably, the cellulose polymers have a degree of substitution on the anhydroglucose unit of from about zero up to and including about 3. As is known to the person skilled in the art the degree of substitution is the average number of hydroxyl groups present on the anhydroglucose unit of the cellulose polymer that are replaced by a substituent group. Suitable cellulose polymers include, but shall not be limited to, polymers selected from cellulose acylate, cellulose diacylate, cellulose triacylate, cellulose acetate, cellulose diacetate, cellulose triacetate, mono-, di-, and tricellulose alkanylates, mono-, di-, and tricellulose aroylates, and mono-, di-, and tricellulose alkenylates. Exemplary cellulose polymers include cellulose acetate having a degree of substitution of from about 1 to about 2 and cellulose acetate having a degree of substitution of from about 2 to about 3. Thus, the cellulose polymer may comprise ethylcellulose, cellulose acetate, cellulose propionate (low, medium, or high molecular weight), cellulose acetate propionate, cellulose acetate butyrate, cellulose acetate phthalate, or cellulose triacetate. An especially preferred cellulose according to this aspect of the invention is ethylcellulose.

More specific cellulose polymers which may be mentioned include cellulose propionate having a degree of substitution of about 1.8; cellulose acetate butyrate having a degree of substitution of about 1.8; cellulose triacylate having a degree of substitution of about 2.9 to 3, such as cellulose triacetate, cellulose trivalerate, cellulose trilaurate, cellulose tripalmitate, cellulose trisuccinate, and cellulose trioctanoate; cellulose diacylates having a degree of substitution of about 2.2 to 2.6 such as cellulose disuccinate, cellulose dipalmitate, cellulose dioctanoate, cellulose dipentanoate, and co-esters of cellulose such as cellulose acetate butyrate, cellulose acetate octanoate butyrate, and cellulose acetate propionate.

The adverse agent-containing core may optionally comprise one or more binders, additional retardants, plasticizers, and/or excipients. Binders may be useful for maintaining the integrity of the matrix and can also help to delay the release of an agent into the bodily fluid. Examples of binders include, but shall not be limited to, natural and synthetic waxes, water insoluble waxes, fatty alcohols, fatty acids, hydrogenated fats, fatty acid esters, fatty acid glycerides, hydrocarbons, and hydrophobic and hydrophilic polymers having hydrocarbon backbones, and mixtures such as, stearyl alcohol, stearic acid, and water soluble polymers such as hydroxycelluloses.

Plasticisers may be useful when the hydrophobic matrix material contains cellulose polymer or an acrylic polymer. Examples of suitable plasticisers include, but shall not be limited to, acetyl triethyl citrate and/or acetyl tributyl citrate.

The adverse agent (antagonist) core may also include other excipients, which can be added to improve the processability of the formulation during extrusion and/or to improve the properties of the final product. Examples of liquid excipients include water and oils, including those of petroleum, animal, vegetable, or synthetic origin, such as peanut oil, soybean oil, mineral oil, sesame oil, castor oil, triglycerides and the like. Examples of solid excipients include magnesium stearate, saline, gum acacia, gelatin, starch paste, talc, keratin, colloidal silica, urea and the like. Colouring agents may also be added to the core.

In a further aspect of the present invention we provide a bioerodable abuse resistant transmucosal drug delivery device and method of treatment using such a device. Such a drug delivery device according to this aspect of the present invention may provide reduced illicit abuse potential. The transmucosal drug delivery device of the present invention may generally include a therapeutic agent (agonist) and its antagonist contained within the device such that abuse of the therapeutic agent is impeded.

Thus, for example, illicit use efforts to extract an abusable drug from the transmucosal devices of the present invention for parenteral injection (e.g., by extraction of the drug by dissolving some or all of the transmucosal device in water or other solvent), can be thwarted by the co-extraction of an antagonist. The amount of antagonist contained in the product is chosen to block any pharmacological effects that would be expected from parenteral administration of the therapeutic agent alone. The antagonist is generally associated with an abuse-resistant matrix, and does not interfere with the transmucosal delivery of the therapeutic agent.

One advantage of the device of this aspect of the present invention is that the device will generally include an abuse-resistant matrix that does not effectively release the antagonist when the device is used in a non-abusive manner. This impairs the activity of the therapeutic agent and it often becomes necessary to increase the quantity thereof required in the dosage form for satisfactory treatment of the patient. The risk of the occurrence of undesirable accompanying symptoms is also increased in comparison to dosage forms which contain no antagonists. Moreover, it is desirable not to further increase the stress on the patient by releasing a large proportion of antagonist when such a dosage form is correctly administered.

One of the advantages of the device of this aspect of the present invention is that the device may be bioerodable, such that the device does not have to be removed after use.

Accordingly, in one aspect, the present invention includes a bioerodable abuse-resistant drug delivery device. The device generally includes transmucosal drug delivery composition and an abuse-resistant matrix. The transmucosal drug delivery composition includes an abusable therapeutic agent (drug) as hereinbefore described and the abuse-resistant matrix includes an antagonist to the abusable therapeutic agent (drug). The delivery device can be, for example, a mucoadhesive drug delivery device, a buccal delivery device, and/or a sublingual delivery device. In one embodiment, the antagonist may be substantially transmucosally unavailable. In other embodiments, the device may be substantially free of inactivating agents.

In another embodiment, the abuse-resistant matrix may be a layer or coating, e.g., a water-erodable coating or layer at least partially disposed about the antagonist. The abuse-resistant matrix may be a water-hydrolysable, water-erodable or water-soluble matrix, e.g., an ion exchange polymer. In one embodiment, the delivery device may be in the form of a tablet, a lozenge, a film, a disc, a capsule or a mixture of polymers.

The device may include a mucoadhesive layer. Furthermore, the device may include a mucoadhesive layer and a non-adhesive backing layer. The device may include a third layer disposed between the mucoadhesive layer and the backing layer. Either or both of the abusable drug and the abuse-resistant matrix are incorporated into a mucoadhesive layer. The abuse-resistant matrix may be incorporated into the backing layer and either or both of the abusable drug and the abuse-resistant matrix may be incorporated into the third layer. The abuse-resistant matrix may be in the third layer and either or both of the abusable drug and the abuse-resistant matrix may be incorporated into any combination of layers as hereinbefore described. Thus, the abusable drug may be incorporated into the mucoadhesive layer and the abuse-resistant matrix may be incorporated into the backing layer.

In an alternative embodiment the abuse-resistant matrix may erode at a rate slower than that of the backing layer, the mucoadhesive layer, the third layer, or any combination thereof.

The abuse-resistant matrix may include, but is not limited to, partially cross linked polyacrylic acid, polycarbophil, povidone, cross-linked sodium carboxymethyl cellulose, gelatin, chitosan, Amberlite™. Duolite™, and combinations thereof. Alternatively, the abuse-resistant matrix may include, but is not limited to, alginates, polyethylene oxide, poly ethylene glycols, polylactide, polyglycolide, lactide-glycolide copolymers, poly-epsilon-caprolactone, polyorthoesters, polyanhydrides and derivatives, methyl cellulose, ethyl cellulose, hydroxypropyl cellulose, hydroxyethyl cellulose, hydroxyethylmethyl cellulose, hydroxypropylmethyl cellulose, polyacrylic acid, and sodium carboxymethyl cellulose, poly vinyl acetate, poly vinyl alcohols, polyethylene glycol, polyethylene oxide, ethylene oxide-propylene oxide co-polymers, collagen and derivatives, gelatin, albumin, polyaminoacids and derivatives, polyphosphazenes, polysaccharides and derivatives, chitin, or chitosan bioadhesive polymers, polyacrylic acid, polyvinyl pyrrolidone, sodium carboxymethyl cellulose, and combinations thereof.

Bioerodable materials according to this aspect of the invention may include, but are not limited to, polymers, copolymers and blends of polyanhydrides (e.g., those made using melt condensation, solution polymerization, or with the use of coupling agents, aromatic acids, aliphatic diacids, amino acids, e.g., aspartic acid and glutamic acid, and copolymers thereof); copolymers of epoxy terminated polymers with acid anhydrides; polyorthoesters; homo- and copolymers of α-hydroxy acids including lactic acid, glycolic acid, ε-caprolactone, γ-butyrolactone, and δ-valerolactone; homo- and copolymers of α-hydroxy alkanoates; polyphosphazenes; polyoxyalkylenes, e.g., where alkene is 1 to 4 carbons, as homopolymers and copolymers including graft copolymers; poly(amino acids), including pseudo poly(amino acids); polydioxanones; and copolymers of polyethylene glycol with any of the above.

In other embodiments, the antagonist and the abusable drug can be combined in a sublingual or buccal monolayer or multilayer tablets. In some embodiments, the antagonist and the abusable drug are incorporated into a mucoadhesive liquid and/or a mucoadhesive solid formulation. It is to be understood that any sublingual tablet, buccal tablet, mucoadhesive liquid formulation and/or mucoadhesive solid formulation can be used with the teachings of the present invention to provide an abuse-resistant device of the present invention.

The antagonist and the abusable therapeutic agent of the present invention may be incorporated into a delivery device such as a transdermal drug device, for example, a transdermal patch.

Alternatively, the abuse-resistant drug delivery device may be in the form of a disc, patch, tablet, solid solution, lozenge, liquid, aerosol or spray or any other form suitable for transmucosal delivery.

In one embodiment of this aspect of the invention, the abusable therapeutic agent may be included in a mucoadhesive layer, generally closest to the treatment site, and the backing layer protects the mucoadhesive layer from contact with saliva or other fluid resulting in slower dissolution of the mucoadhesive layer and longer contact of the mucoadhesive layer and drug with the treatment site. In such embodiments, the placement of the abusable drug in the mucoadhesive layer allows the abusable therapeutic agent to unidirectionally diffuse through the buccal mucosa of the mouth and into the systemic circulation, while avoiding first pass metabolism by the liver.

The mucoadhesive layer, e.g., a bioerodible mucoadhesive layer, may generally comprise a water soluble polymer which includes, but shall not be limited to, hydroxyethyl cellulose, hydroxypropyl cellulose, hydroxypropylmethyl cellulose, hydroxyethylmethyl cellulose, polyacrylic acid which may or may not be partially cross linked, sodium carboxymethyl cellulose, and polyvinylpyrrolidone or combinations thereof. Other mucoadhesive water-soluble polymers may also be used in the present invention.

The backing layer, e.g., a bioerodible non-adhesive backing layer, may generally comprise a water-soluble, film-forming pharmaceutically acceptable polymers which include, but are not limited to, hydroxyethyl cellulose, hydroxypropyl cellulose, hydroxypropylmethyl cellulose, hydroxyethylmethyl cellulose, polyvinyl alcohol, polyethylene glycol, polyethylene oxide, ethylene oxide-propylene oxide co-polymers, or combinations thereof. The backing layer may comprise other water-soluble, film-forming polymers as known in the art.

Any of the layers in the devices of the present invention may also contain a plasticising agent, such as propylene glycol, polyethylene glycol, or glycerin in a small amount, 0 to 15% by weight, in order to improve the “flexibility” of this layer in the mouth and to adjust the erosion rate of the device. In addition, humectants such as hyaluronic acid, glycolic acid, and other alpha hydroxyl acids can also be added to improve the “softness” and “feel” of the device. Colourants and opacifiers may be added to help distinguish the resulting non-adhesive backing layer from the mucoadhesive layer. Some opacifiers which may be mentioned include titanium dioxide, zinc oxide, zirconium silicate, etc.

The abuse-resistant matrix includes materials used for chemical binding, e.g., in ion-exchange polymers. Such materials include, but are not limited to, polyanhydrides, poly(hydroxyethyl methacrylate), polyacrylic acid, sodium acrylate, sodium carboxymethyl cellulose, poly vinyl acetate, poly vinyl alcohols, poly(ethylene oxide), ethylene oxide-propylene oxide co-polymers, poly(N-vinyl pyrrolidone), poly(methyl methacrylate), polyacrylamide, poly(ethylene-co-vinyl acetate), poly(ethylene glycol), poly(methacrylic acid), gelatin, chitosan, collagen and derivatives, albumin, polyaminoacids and derivatives, polyphosphazenes, polysaccharides and derivatives thereof.

In one embodiment, the abuse-resistant matrix may be a layer coating, e.g., a water-erodable coating. That is, physical entrapment of the antagonist in the device, e.g., the mucoadhesive layer, can be facilitated by a barrier layer which is coated with a water soluble polymer which erodes slowly. The antagonist may be at least partially coated or disposed within water-erodible coating.

The abuse-resistant matrix may include materials used for physical entrapment, such as, alginates, polyethylene oxide, poly ethylene glycols, polylactide, polyglycolide, lactide-glycolide copolymers, poly-epsilon-caprolactone, polyorthoesters, polyanhydrides and derivatives, methyl cellulose, ethyl cellulose, hydroxypropyl cellulose, hydroxyethyl cellulose, hydroxyethylmethyl cellulose, hydroxypropylmethyl cellulose, polyacrylic acid, and sodium carboxymethyl cellulose, poly vinyl acetate, poly vinyl alcohols, polyethylene glycol, polyethylene oxide, ethylene oxide-propylene oxide co-polymers, collagen and derivatives, gelatin, albumin, polyaminoacids and derivatives, polyphosphazenes, polysaccharides and derivatives, chitin, chitosan bioadhesive polymers, polyacrylic acid, polyvinyl pyrrolidone, sodium carboxymethyl cellulose and combinations thereof.

The invention will now be illustrated by way of example only.

### EXAMPLE 1

Phase II Clinical Study

A single centre, double blind, non-inferiority study was conducted to evaluate the antidepressant activity of Viotra™ (extended release tramadol hydrochloride) compared with amitriptyline in the treatment of major depressive disorder (MDD) in patients who have an unsatisfactory response/are resistant to SSRIs.

Objectives:

Primary Objective

To demonstrate that the antidepressant activity of Viotra™ is not inferior to amitriptyline in subjects who have an unsatisfactory response to/are resistant to treatment with SSRIs.

Secondary

To evaluate the safety and tolerability of Viotra™.

Study Design:

A phase II single centre double blind, non-inferiority, parallel, dose response study.

After informed consent has been obtained, details of demography, medical and psychiatric history and prior and current medication will be recorded in the case report form. Eligibility criteria will be checked including the 17-item HAM-D scale and the MINI assessment. A physical examination will be carried out and vital signs, weight and a 12-lead electrocardiogram (ECG) recorded. Blood and urine samples will be taken for routine haematology and clinical chemistry tests. A pregnancy test will be performed where appropriate.

After screening, subjects will start a lead in phase of 4 weeks treatment beginning with 10 mg paroxetine in week 1 and increasing to 20 mg paroxetine/day for weeks 2-4. Subjects must have a HAMD-17 score of ≧18 at the start of the lead in phase. Subjects who have a HAMD-17 score of ≧16 at the end of the 4 weeks will be randomised to one of three treatment groups (Week 0). Subjects will take 20 mg tramadol or 70 mg tramadol or 75 mg amitriptyline once daily in the evening over 8 weeks.

Test Product, Dose and Mode of Administration:

Viotra™: Active substance: extended release tramadol hydrochloride.

Dose: 20 mg tramadol (Group 1) taken once daily orally with water at between 7-9 p.m. (evening).

or

70 mg tramadol (Group 2) taken once daily orally with water at approximately 7-9 p.m. (evening).

Reference Product, Dose and Mode of Administration:

Active substance: amitriptyline.

Dose: 75 mg tablet, taken once daily orally with water at between 7-9 p.m. (evening).

Efficacy Endpoints

Primary Efficacy Endpoint:
The mean difference in baseline-adjusted MADRS score at the end of treatment between two doses of Viotra™ and amitriptyline.

Secondary efficacy endpoints:
The mean difference in baseline-adjusted MADRS score at weeks 1, 2, 4 and 6 between two doses of Viotra™ and amitriptyline.
Percentage of subjects with remission defined as ≦10 on the MADRS at the end of treatment with Viotra™ or amitriptyline.
Percentage of responders defined as ≧50% decrease from baseline the MADRS at the end of treatment with Viotra™ or amitriptyline.
Percentage of partial responders defined as <50% and ≧25% decrease from baseline depression on the MADRS at the end of treatment with Viotra™ or amitriptyline.
The mean difference in baseline-adjusted CGI severity at weeks 1, 2, 4, 6 and 8.
The mean difference in baseline-adjusted CGI improvement at weeks 1, 2, 4, 6 and 8.

What is claimed is:

1. A method of treatment of a patient suffering from depression, said method comprising the administration of a sub-analgesic amount of tramadol, or a salt thereof.

2. A method of treatment according to claim 1 which comprises the once daily administration of a sub-analgesic amount of tramadol, or a salt thereof.

3. A method of treatment according to claim 1 which comprises the treatment, alleviation or prevention of depression.

4. A method of treatment according to claim 1 wherein the amount of tramadol, or a salt thereof, comprises <100 mg daily dose.

5. A method of treatment according to claim 1 wherein the amount of tramadol, or a salt thereof, comprises ≧10 mg daily dose.

6. A method of treatment according to claim 1 wherein the amount of tramadol, or a salt thereof, comprises from ≧10 mg to <100 mg daily dose.

7. A method of treatment according to claim 1 wherein depression is selected from the group comprising major depression, chronic mild depression, manic depression (bipolar disorder), atypical depression, psychotic depression and dysthymia.

8. A method of treatment according to claim 1 wherein the patient is one of the group of patients who show no or insufficient response to the use of SSRIs.

9. A method of treatment according to claim 1 in which the sub-analgesic amount of tramadol, or a salt thereof, upon initial administration, provides an onset of antidepressive effect within 2 hours, which antidepressive effect continues for at least 24 hours after administration.

10. A method of treatment according to claim 1 comprising a sub-analgesic amount of tramadol, or a salt thereof, in combination with a second therapeutically active ingredient.

11. A pharmaceutical composition comprising a sub-analgesic amount of tramadol, or a salt thereof, in association with a pharmaceutically acceptable adjuvant, diluent or carrier.

12. A pharmaceutical composition according to claim 11 for use in the treatment, alleviation or prevention of depression.

13. A pharmaceutical composition according to claim 11 wherein the amount of tramadol, or a salt thereof, comprises <100 mg daily dose.

14. A pharmaceutical composition according to claim 11 wherein the amount of tramadol, or a salt thereof, comprises ≧10 mg daily dose.

15. A pharmaceutical composition according to claim 11 wherein the amount of tramadol, or a salt thereof, comprises from ≧10 mg to <100 mg daily dose.

16. A pharmaceutical composition according to claim 11 wherein depression is selected from the group comprising major depression, chronic mild depression, manic depression (bipolar disorder), atypical depression, psychotic depression and dysthymia.

17. A pharmaceutical composition according to claim 11 wherein the patient is one of the group of patients who show no or insufficient response to the use of SSRIs.

18. A pharmaceutical composition according to claim 11 in which the composition, upon initial administration, provides an onset of antidepressive effect within 2 hours, which antidepressive effect continues for at least 24 hours after administration.

19. A pharmaceutical composition according to claim 11 comprising a sub-analgesic amount of tramadol, or a salt thereof, in combination with a second therapeutically active ingredient.
